(19) United States
(12) Patent Application Publication
(10) Pub. No.: US 2022/0257357 A1
(43) Pub. Date: Aug. 18, 2022

(54) Multi-Aperture Aerosol Extractor and Retractor

(71) Applicants: Christopher J. Root, Belchertown, MA (US); Beata Root, Belchertown, MA (US)

(72) Inventors: Christopher J. ROOT, Belchertown, MA (US); Beata Root, Belchertown, MA (US); Madison Shultz, Watertown, MA (US); Jeffrey Eerier, Franklin, MA (US)

(21) Appl. No.: 17/669,182

(22) Filed: Feb. 10, 2022

Related U.S. Application Data
(60) Provisional application No. 63/150,672, filed on Feb. 18, 2021.

(51) Int. Cl. A61C 17/10; A61C 17/08

(52) U.S. Cl. CPC A61C 17/10; A61C 17/08

(57) ABSTRACT

A device, and method of using same, to evacuate aerosols generated proximate to teeth of a patient during dental procedures, including a central housing having a proximal end and a distal end. The proximal end defines a proximal housing opening and the distal end defines a distal housing opening having a first open area. The housing defines (i) at least one housing side port on each side of the housing and (ii) at least one internal passage in fluid communication with the distal housing opening, the proximal housing opening, and the housing side ports. The proximal end of the housing is shaped to be connectable to a conduit for a source of negative pressure. The device further includes a right-side cheek retractor and a left-side cheek retractor, each retractor defining a concave buccal-side surface, a convex oral-side surface, a retractor chamber, and at least a first arc of retractor openings (i) positioned along a portion of the oral-side surface, (ii) communicating with the retractor chamber, and (iii) having a first distribution of total open area along that retractor. A right-side arm extends between the housing and the right-side cheek retractor to support the right-side cheek retractor at a substantially fixed distance from the housing, and a left-side arm extends between the housing and the left-side cheek retractor to support the left-side cheek retractor at a substantially fixed distance from the housing. At least one right-side passageway connects one of the housing side ports with the retractor chamber of the right-side cheek retractor, and at least one left-side passageway connecting one of the housing side ports with the retractor chamber of the left-side cheek retractor.

### CROSS-REFERENCE TO RELATED APPLICATION

This application claims priority to U.S. Provisional Application No. 63/150,672 filed on 18 Feb. 2021. The entire contents of the above-mentioned application are incorporated herein by reference.

### FIELD OF THE INVENTION

This invention relates to retractors for dentistry and more particularly to combination devices which also provide multi-port aspiration.

### BACKGROUND OF THE INVENTION

A common challenge in dentistry is to provide a dry-field isolation of the operative field. The lips, cheeks, and tongue often interfere with good dental operative technique. In addition, the mouth is full of saliva, operative debris, fluid, and aerosol which must be removed. Most importantly, the oral cavity is a reservoir for bacteria, viruses, and oral pathogens. With the advent of the COVID-19 virus, infection control and aerosol management has moved to the forefront of dentistry.

Many dental devices have been invented in attempts to solve the oral tissue retraction issue. A combined mouth retractor and saliva remover is disclosed by Gelarie in U.S. Pat. No. 3,455,024. Rhoades discloses a jaw rest and ejector in U.S. Pat. No. 4,802,851. Schoolman et al. describe a collector placed around the exterior of lips of a patient. An oral isolation device with evacuation chambers is discussed by Costello in U.S. Pat. No. 6,213,772. Boada et al. describe an auxiliary dental device in U.S. Patent Application Pub. No. 2019/0365214A1. However, the oral aerosol evacuation issue has not been adequately addressed by such devices.

Dental offices are already well-equipped in high-volume evacuation (“HVE”) in the form of conventional saliva ejectors. However, using only a saliva ejector is not sufficient in aerosol control. The ability to catch and remove aerosols at the source makes the whole office a safer and healthier place for team members and patients. Putting together both tissue retraction and aerosol control would be a very successful tool for aerosol control. Dental professionals play an important role in the prevention of transmission of COVID-19. Coronaviruses are airborne viruses. Many studies show that the ultrasonic scaler can be the most dangerous aerosol emission producer. In addition, many dental hygienists work solo and are not able to use HVE system by themselves. The use of HVE has been shown to reduce contamination at the operative site by more than 90%.

Some known devices serve both as a cheek retractor and an HVE source, but with limitations. Some devices contain only one large HVE port at the base of each device. Many studies have indicated that single-source HVE is inadequate for aerosol management.

Numerous studies have shown that using only one HVE opening in a device (“pinpoint HVE suction”) does not provide adequate aerosol control. These studies also show that having HVE located 6-15 mm from the work zone, the source of aerosol, can provide adequate aerosol control. “Pinpoint HVE suction,” through a single orifice, leaves large areas of the oral cavity without HVE coverage, as most molars being treated by the dentist are outside of the 6-15 mm window of coverage.

There are benefits to utilizing a single-use device which does not cause any damage to the oral cavity or to the surrounding tissue. A single-use device provides patient safety and results in no exposure to pathogens via cross-contamination among patients. No maintenance or cleaning of the single-use device is required.

It is therefore desirable to create a device which is multi-faceted, one that provides mechanical retraction of the oral tissues and also removes pathogen-laden aerosols and fluids.

### SUMMARY OF THE INVENTION

An object of the present invention is to provide a multi-purpose device which serves as a lip and cheek retractor, optimizes the size and isolation of the operating field, and successfully evacuates aerosol, fluids, and other operative debris.

Another object of the invention is to keep the operative field dry without causing damage or irritation to oral tissues.

Yet another object of the invention is to enable a single practitioner, working solo, to effectively control air quality at the source during dental work.

This invention results from the realization that arranging multiple HVE (“high-speed evacuation” or “high-volume evacuation”) holes around the periphery of a cheek retractor portion of an improved dental device can effectively control aerosols while utilizing a single conventional source of negative pressure. It is the size, distribution, and arrangement of these HVE holes which provide the significant reduction of aerosols and serve as a key differentiating factor from previous designs. The present invention utilizes “broad sweep suction” as the most effective aerosol mitigation method and achieves optimum air control by installing openings substantially all around the periphery of the device. By arranging these openings in various sizes, such as from smallest to largest size approaching a central distal opening, we can achieve equalized suction around the entire oral cavity—not just in one location. Preferably, there will be no portion of the oral cavity left outside of an optimum 6-15 mm window of coverage.

This invention features a device, and method of using same, suitable for evacuating aerosols generated proximate to teeth of a patient during dental procedures, including a central housing having a proximal end and a distal end. The proximal end defines a proximal housing opening and the distal end defines a distal housing opening having a first open area. The housing defines (i) at least one housing side port on each side of the housing and (ii) at least one internal passage in fluid communication with the distal housing opening, the proximal housing opening, and the housing side ports. The proximal end of the housing is shaped to be connectable to a conduit for a source of negative pressure. The device further includes a right-side cheek retractor and a left-side cheek retractor, each retractor defining a concave buccal-side (cheek-side) surface, a convex oral-side (tongue-side) surface, a retractor chamber, and at least a first arc of retractor openings (i) positioned along a portion of the oral-side surface, (ii) communicating with the retractor chamber, and (iii) having a first distribution of total open area along that retractor. A right-side arm extends between the housing and the right-side cheek retractor to support the right-side cheek retractor at a substantially fixed right-side distance from the housing, and a left-side arm extends between the housing and the left-side cheek retractor to support the left-side cheek retractor at a substantially fixed left-side distance from the housing. At least one right-side passageway connects one of the housing side ports with the retractor chamber of the right-side cheek retractor, and at least one left-side passageway connecting one of the housing side ports with the retractor chamber of the left-side cheek retractor.

In some embodiments, at least one of the right-side passageway and/or the left-side passageway is defined by tubing such as flexible polymeric tubing which is placeable proximate to the respective arm. In other embodiments, at least one of the right-side passageway and/or the left-side passageway is defined by the right-side arm and/or the left-side arm, respectively; in other words, at least one of the arms may be hollow or otherwise contain the respective passageway.

In certain embodiments, (i) the oral-side surface of each cheek retractor includes (a) an inner portion positionable toward the pharynx of the patient, (b) an outer portion positionable toward a practitioner, and (c) a middle portion disposed between the inner portion and the outer portion, and (ii) the first arc of retractor openings extends along at least one of the outer portion and the middle portion of the oral-side surface of each cheek retractor. In a number of embodiments, at least one of the arcs of retractor openings has openings that occur in a selected progression, such as decreasing in size progressing away from the distal housing opening, such as an arc of “descending” openings having progressively smaller open areas. In some embodiments, the device further includes a second arc of retractor openings positionable closer to the pharynx of the patient and having a second distribution of total open area along that retractor that is smaller than the first distribution of total open area along that retractor. In one embodiment, the second arc of retractor openings extends along the inner portion of the oral-side surface of each cheek retractor

This invention further features a method of evacuating aerosols including selecting a device as described above, positioning the device such that the buccal-side surface of the right-side cheek retractor contacts a left cheek of the patient and the buccal-side surface of the left-side cheek retractor contacts a right cheek of the patient, with the distal housing opening positioned below the mouth of the patient, and applying negative pressure to the proximal housing opening to evacuate aerosols, generated proximate to the teeth of the patient during a dental procedure, through the retractor openings and the distal housing opening.

### BRIEF DESCRIPTION OF THE DRAWINGS

In what follows, preferred embodiments of the invention are explained in more detail with reference to the drawings, in which:

FIG. 1A is a schematic practitioner-side perspective view of a device according to the present invention;

FIG. 1B is a view similar to FIG. 1A with fluid passages indicated in phantom;

FIG. 2A is a schematic bottom or patient-side view of the device of FIG. 1A;

FIG. 3A is a schematic rear view of the device of FIG. 1A showing the proximal end of the central housing;

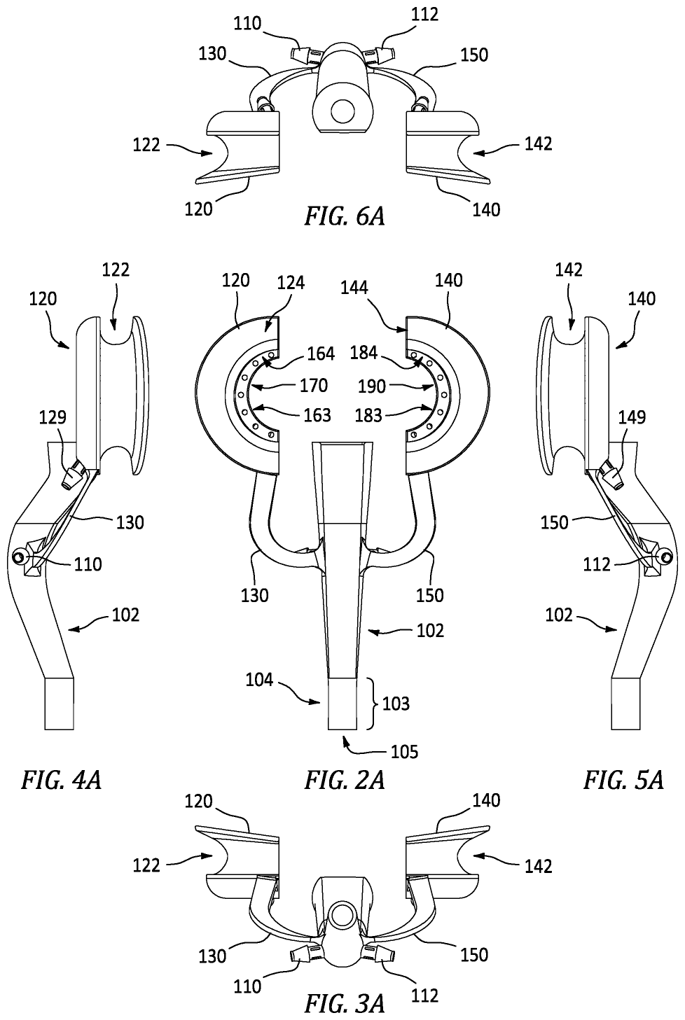

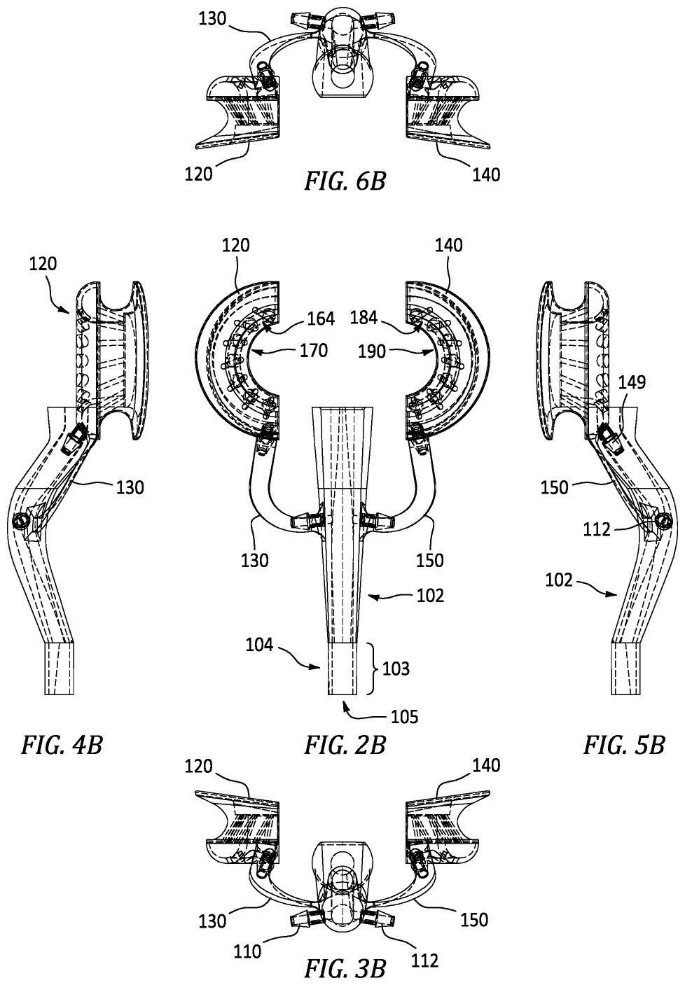

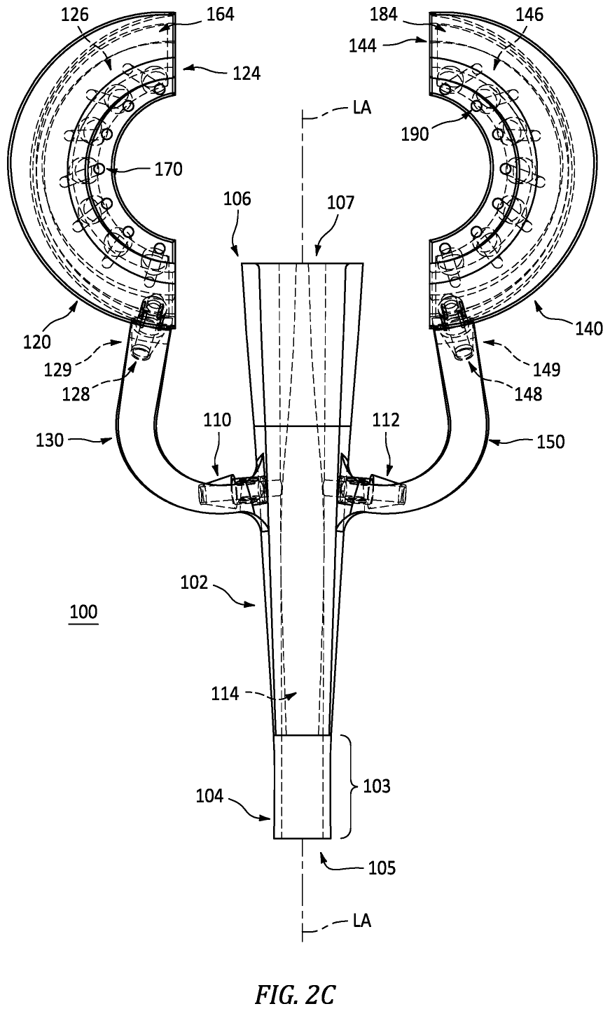

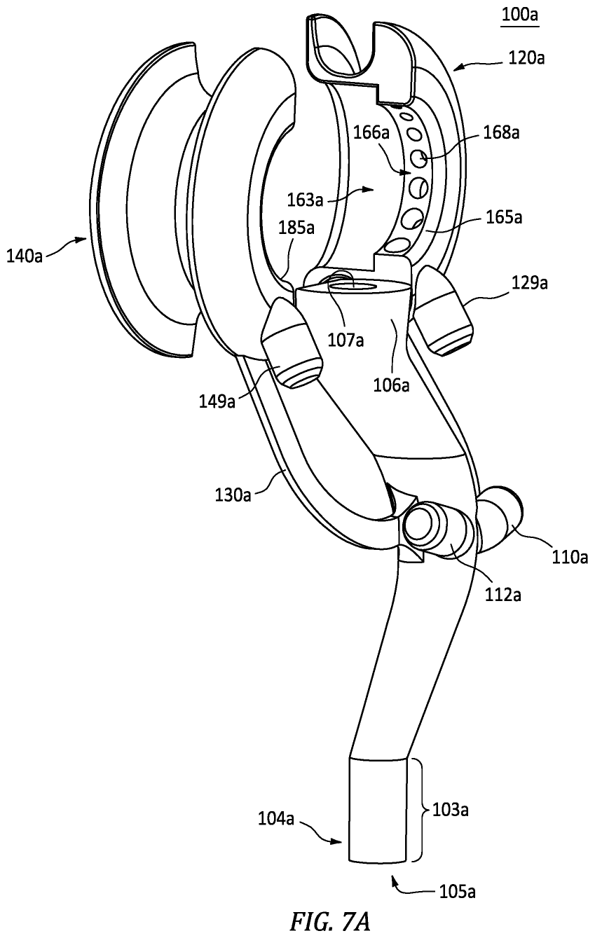

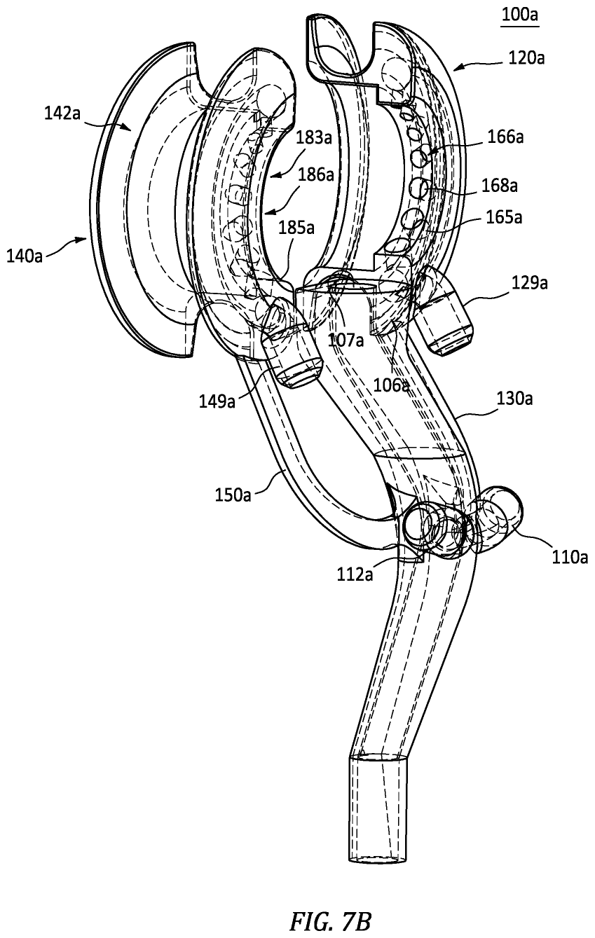

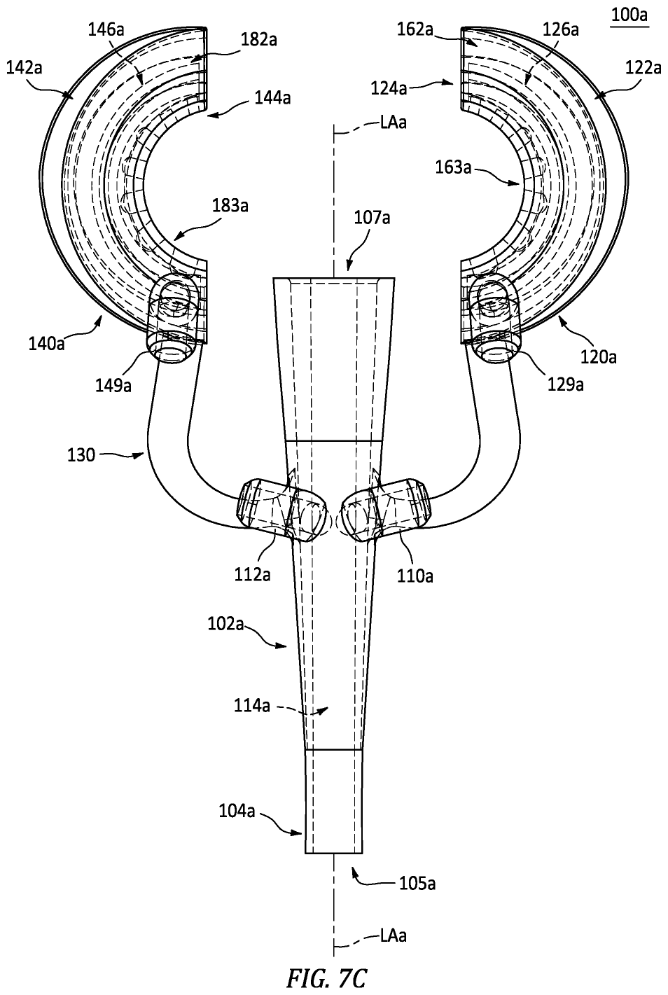

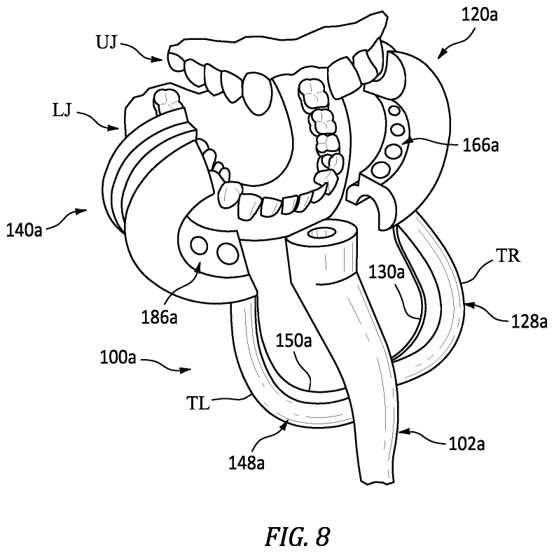

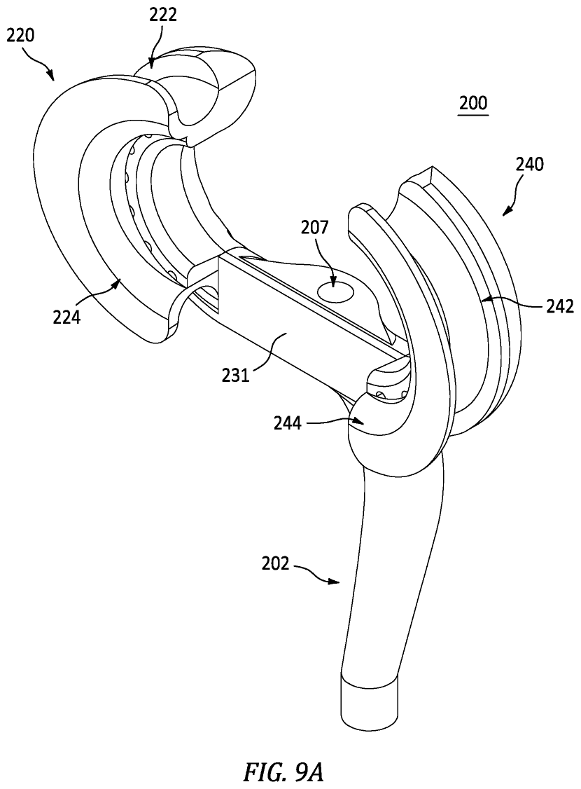

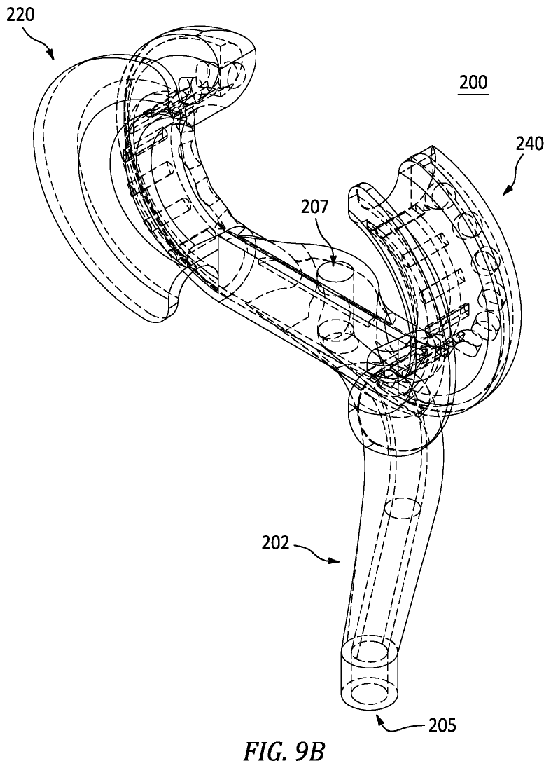

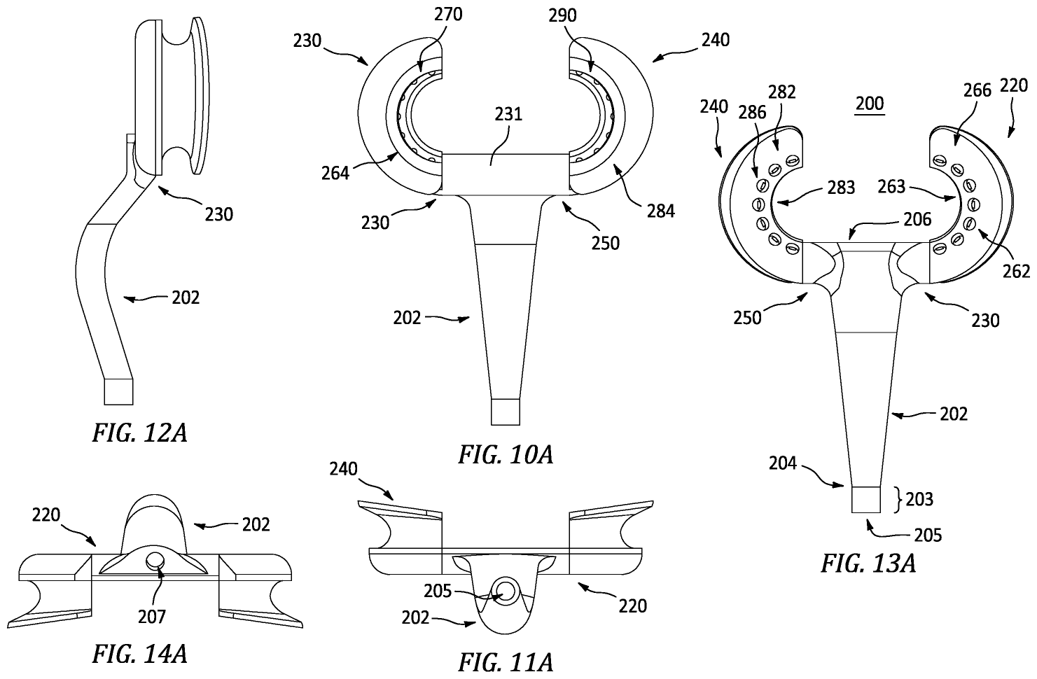

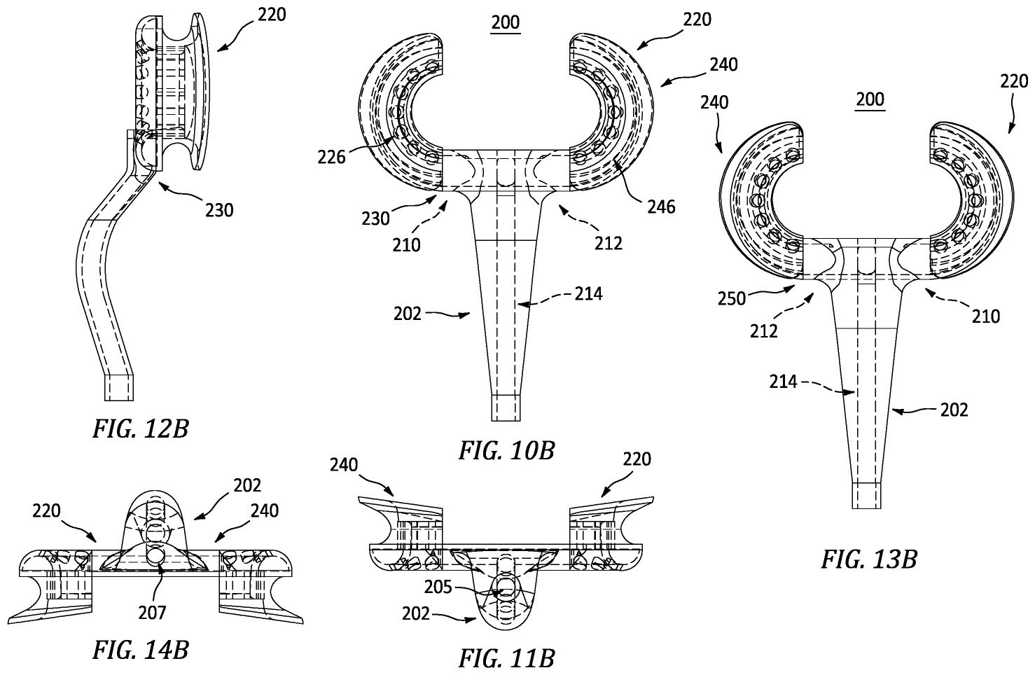

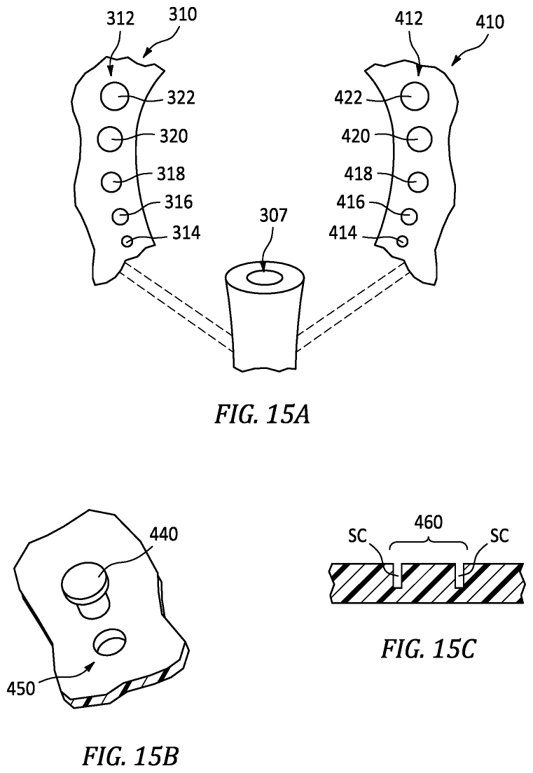

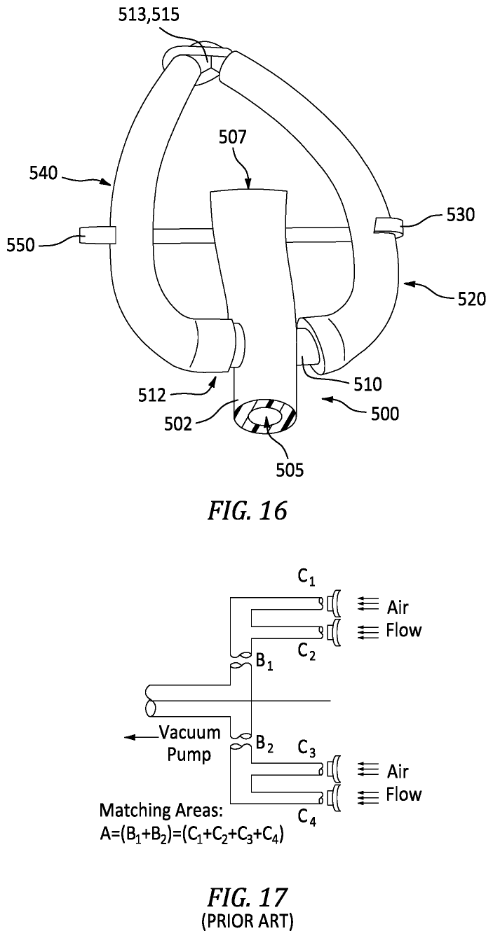

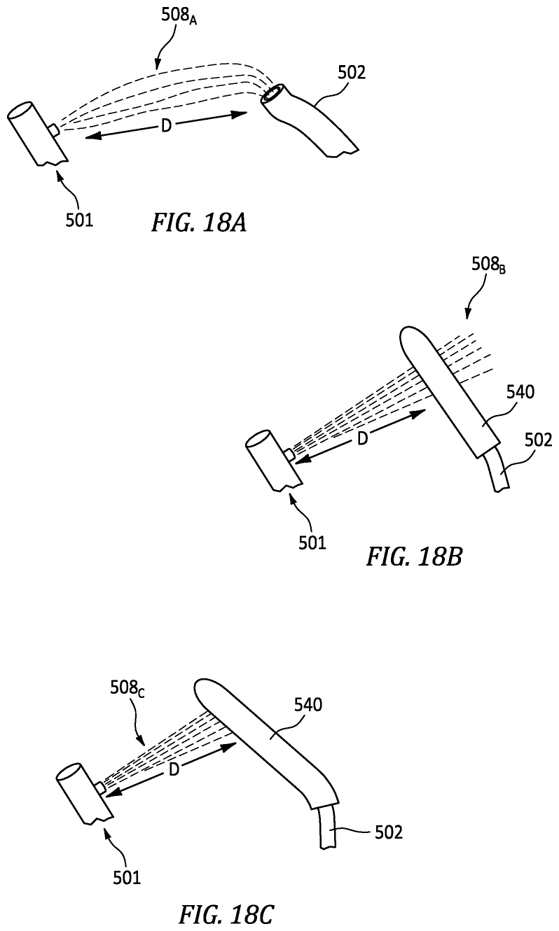

FIGS. 2B and 3B are views similar to FIGS. 2A and 3A with fluid passages indicated in phantom;

FIG. 2C is an enlargement of FIG. 2B, which is a patient-side view;

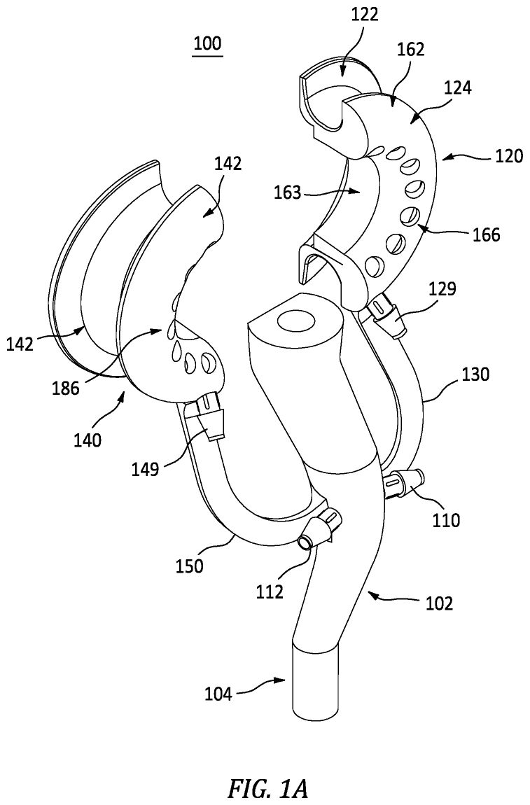

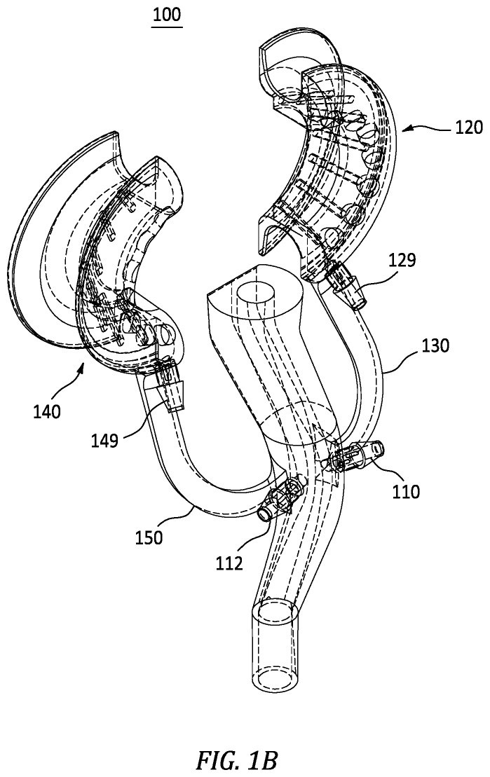

FIGS. 4A and 5A are schematic right side and left side views of the device of FIG. 1A, respectively;

FIGS. 4B and 5B are views similar to FIGS. 4A and 5A with fluid passages indicated in phantom;

FIG. 6A is a schematic front view of the device of FIG. 1A showing the distal end of the housing;

FIG. 6B is a view similar to FIG. 6A with fluid passages indicated in phantom;

FIG. 7A is a schematic practitioner-side perspective view of an alternative embodiment of a device according to the present invention;

FIG. 7B is a view similar to FIG. 7A with fluid passages indicated in phantom;

FIG. 7C is a schematic practitioner-side top view of the device of FIGS. 7A-7B showing fluid passages in phantom;

FIG. 8 is a schematic perspective practitioner-side view of the device of FIGS. 7A-7C in use relative to jaws and teeth of a patient;

FIG. 9A is a schematic patient-side perspective view of another embodiment of a device according to the present invention;

FIG. 9B is a view similar to FIG. 9A with fluid passages indicated in phantom;

FIGS. 10A and 11A are schematic bottom (patient-side) and rear views of the device of FIG. 9A, respectively;

FIGS. 10B and 11B are views similar to FIGS. 10A and 11A with fluid passages indicated in phantom;

FIG. 12A is a schematic right side view of the device of FIG. 9A;

FIG. 13A is a schematic practitioner-side view of the device of FIG. 9A;

FIGS. 12B and 12B are views similar to FIGS. 12A and 14A with fluid passages indicated in phantom;

FIG. 14A is a schematic front view of the device of FIG. 9A;

FIG. 14B is a view similar to FIG. 14A with fluid passages indicated in phantom;

FIG. 15 is a schematic representation of a device according to the present invention having a left-side retractor defining an arc of “ascending” openings having progressively larger open areas and a right-side retractor defining an arc of “descending” openings having progressively, smaller open areas;

FIG. 15B is a schematic perspective view of a plug being inserted into an opening in a cheek retractor to selectively occlude that opening;

FIG. 15C is a schematic cross-sectional view of a punch-out to enable selective use of a potential opening;

FIG. 16 is a perspective, partial cross-sectional, practitioner-side view of a central housing connected to a pair of tubing to represent cheek retractor openings according to the present invention for testing purposes;

FIG. 17 is a schematic cross-sectional graphic representation of known preferred relationships among cross-sectional areas of branching passageways;

FIG. 18A is a schematic side view of a continuous aerosolizer emitting aerosol toward the central housing of FIG. 16 spaced at a distance D and without tubing to simulate a conventional HVE device; and

FIGS. 18B and 18C are schematic side views similar to FIG. 18A, with tubing, FIG. 16, having ascending openings shown in FIG. 18B and having descending openings shown in FIG. 18C.

### DETAILED DESCRIPTION OF THE PRESENTLY PREFERRED EMBODIMENTS

This invention may be accomplished by a device, and method of using same, suitable for evacuating aerosols generated proximate to teeth of a patient during dental procedures, including a central housing having a proximal end and a distal end. The proximal end defines a proximal housing opening and the distal end defines a distal housing opening having a first open area. The housing defines (i) at least one housing side port on each side of the housing and (ii) at least one internal passage in fluid communication with the distal housing opening, the proximal housing opening, and the housing side ports. The proximal end of the housing is shaped to be connectable to a conduit for a source of negative pressure. The device further includes a right-side cheek retractor and a left-side cheek retractor, each retractor defining a concave buccal-side surface, a convex oral-side surface, a retractor chamber, and at least a first arc of retractor openings (i) positioned along a portion of the oral-side surface, (ii) communicating with the retractor chamber, and (iii) having a first distribution of total open area along that retractor. A right-side arm extends between the housing and the right-side cheek retractor to support the right-side cheek retractor at a substantially fixed right-side distance from the housing, and a left-side arm extends between the housing and the left-side cheek retractor to support the left-side cheek retractor at a substantially fixed left-side distance from the housing. At least one right-side passageway connects one of the housing side ports with the retractor chamber of the right-side cheek retractor, and at least one left-side passageway connecting one of the housing side ports with the retractor chamber of the left-side cheek retractor.

The term “effective” as utilized herein refers to an actual parameter experienced during use of a device according to the present invention, such as an “effective barrier” that prevents at least ninety-five percent (95%) of aerosols from escaping past the device during dental procedures.

The term “evacuation” includes aspiration by negative pressure of gases, liquids including blood and saliva, debris including pieces of dental plaque and bodily tissues loosened during hygienic and/or surgical procedures, and aerosols. The rate of evacuation, also referred to as “airflow”, is typically measured in CFM (cubic feet per minute).

The term “HVE source” as utilized herein refers to a source of negative pressure that can withdraw by evacuation at least one-hundred (100) CFM (cubic feet per minute) of air during HVE (“high-speed evacuation” or “high-volume evacuation”) operation.

The terms “left” and “right” as utilized herein refers to an orientation of a device according to the present invention as viewed by a practitioner. For example, a “left-side cheek retractor” will engage the right cheek of a patient.

The term “open area” as utilized herein refers to the overall orifice size of a single opening or of a set of openings, depending on context, and includes the area of irregularly-shaped and non-circular openings including holes and apertures through which air and aerosols may pass.

The term “practitioner” as utilized herein includes healthcare professionals such as dental hygienists and/or persons having a degree of Bachelor of Dental Surgery, Doctor of Dental Medicine, Doctor of Dental Surgery, and/or Doctor of Medicine.

The term “substantially” as utilized herein encompasses deviations of up to ten percent, such as a retractor being at “a substantially fixed distance from the housing” encompassing deviations up to ten percent distance from each other. As another example, “substantially facing a longitudinal axis” encompasses angles of 80 degrees up to 100 degrees, that is, plus or minus ten degrees, relative to a radial line or a lateral line that is perpendicular to that longitudinal axis, or relative to a plane containing that longitudinal axis.

Device 100, FIGS. 1A-6B, has a central housing 102 having a proximal end 104 and a distal end 106. The proximal end 104 defines a proximal housing opening 105 and the distal end 106 defines a distal housing opening 107 having a first open area. The housing 102 defines (i) at least one housing side port 110, 112 on each side of the housing and (ii) at least one internal passage 114 in fluid communication with the distal housing opening 107, the proximal housing opening 105, and the housing side ports 110, 112. The proximal end 104 of the housing is shaped to be connectable, such as by having an outer cylindrical sleeve surface 103, to a conduit for a source of negative pressure such as a vacuum pump unit to serve as a HVE source.

The device 100 further includes a right-side cheek retractor 120 and a left-side cheek retractor 140. Each retractor 120, 140 defines a concave buccal-side surface 122, 142, respectively, and a convex oral-side surface 124, 144, respectively, also referred to herein as lingual-side surface 124, 144 because it is closer to the tongue of the patient. The oral-side surface 124, 144 of each cheek retractor includes an inner portion 164, 184 positionable toward the pharynx of the patient, an inwardly-facing middle portion 163, 183, and an outer portion 162, 182, respectively, positionable toward a practitioner. Middle portions 163, 183 substantially face each other and face a longitudinal axis LA, FIG. 2C, that is, each middle portion lies substantially in a plane that contains longitudinal axis LA and distal housing opening 107.

Each retractor 120, 140 defines a retractor chamber 126, 146, such as shown in dashed lines in FIG. 2C, and at least a first arc 166, 186 of retractor openings (i) positioned along one of the portions 162, 163, 164 and 182, 183, 184 of the oral-side surface 124, 144, (ii) communicating with the retractor chamber 126, 146, and (iii) having a first distribution of total open area along that retractor 120, 140, respectively.

A right-side arm 130 extends between the housing 102 and the right-side cheek retractor 120 to support the right-side cheek retractor 120 at a substantially fixed right-side distance from the housing 102, and a left-side arm 150 extends between the housing 102 and the left-side cheek retractor 140 to support the left-side cheek retractor 140 at a substantially fixed left-side distance from the housing 102. In some constructions, the left-side and right-side fixed distances are substantially the same distance. At least one right-side passageway similar to passageway 128a, FIG. 8, connects the housing side port 110, FIGS. 1A-6B, with connector 129 for the retractor chamber 126 of the right-side cheek retractor 120, and at least one left-side passageway similar to passageway 148a, FIG. 8, connects the housing side port 112, FIGS. 1A-6B, with the retractor chamber 146 of the left-side cheek retractor 140. In this construction, separate tubing (see FIG. 8 for right and left tubing TR, TL) defines a portion of the passageways 128a, 148a. In other constructions, such as described below in relation to FIGS. 9A-14B, the passageways are integral with at least one arm.

In certain constructions, the first arc 166, 186 of retractor openings 168, 188 extends along the outer portion 162, 182 of the oral-side surface 124, 144 of each cheek retractor 120, 140. In other constructions, the first arc of retractor openings extends along the middle portion, such as described below for arcs 163a, 183a, FIGS. 7A-8. In some constructions, the device further includes a second arc 170, 190 of retractor openings 172, 192 positionable closer to the pharynx of the patient and having a second distribution of total open area along that retractor that is smaller than the first distribution of total open area along that retractor. In one embodiment, the second arc 170, 190 of retractor openings extends along the inner portion 164, 184 of the oral-side surface 124, 144 of each cheek retractor 120, 140. In yet other constructions, holes of an arc alternative between two portions, such as alternating between the outer portion and the middle portion of at least one cheek retractor.

Suitable materials for housing 102 and retractors 120, 140 include single-use, biocompatible polyethylene and polycarbonate materials, especially if materials for retractors 120, 140 and arms 130, 150 are formulated to be substantially resilient to return quickly to original shape after force is applied, especially to resist bending of support arms relative to the central housing such that cheeks of a patient remain retracted throughout a dental procedure.

FIGS. 7A-8 illustrate an alternative device 100a according to the present invention having raised, inwardly-facing middle sections 165a, 185a of middle portions 163a, 183a, respectively, to define arcs 166a and 186a of openings. Device 100a has a central housing 102a having a proximal end 104a and a distal end 106a. The proximal end 104a defines a proximal housing opening 105a and the distal end 106a defines a distal housing opening 107a having a first open area. The housing 102a defines (i) at least one housing side port 110a, 112a on each side of the housing and (ii) at least one internal passage 114a in fluid communication with the distal housing opening 107a, the proximal housing opening 105a, and the housing side ports 110a, 112a. The proximal end 104a of the housing is shaped to be connectable, such as by having an outer cylindrical sleeve surface 103a, to a conduit for a source of negative pressure, particularly to a HVE source.

The device 100a further includes a right-side cheek retractor 120a and a left-side cheek retractor 140a. Each retractor 120a, 140a defines a concave buccal-side surface 122a, 142a, respectively, and a convex oral-side surface 124a, 144a. The oral-side surface 124a, 144a of each cheek retractor includes an inner portion 164a, 184a positionable toward the pharynx of the patient, a middle portion 163a, 183a, and an outer portion 162a, 182a, respectively, positionable toward a practitioner. Middle portions 163a, 183a substantially face a longitudinal axis LAa, FIG. 7C, that is, each middle portion lies substantially in a plane containing that longitudinal axis LAa and distal housing opening 107a.

In this construction, each retractor 120a, 140a defines a retractor chamber 126a, 146a, such as shown in dashed lines in FIG. 7C, and only a first arc 166a, 186a of retractor openings (i) positioned along only middle portion 163a and 183a of the oral-side surface 124a, 144a, (ii) communicating with the retractor chamber 126a, 146a, and (iii) having a first distribution of total open area along that retractor 120a, 140a, respectively.

A right-side arm 130a extends between the housing 102a and the right-side cheek retractor 120a to support the right-side cheek retractor 120a at a substantially fixed right-side distance from the housing 102a, and a left-side arm 150a extends between the housing 102a and the left-side cheek retractor 140a to support the left-side cheek retractor 140a at a substantially fixed left-side distance from the housing 102a. At least one right-side passageway connects the housing side port 110a with connector 129a for the retractor chamber 126a of the right-side cheek retractor 120a, and at least one left-side passageway connects the housing side port 112a with the retractor chamber 146a of the left-side cheek retractor 140a. In this construction, the right-side passageway and the left-side passageway are defined by separate right and left tubing TR, TL, FIG. 8, such as flexible polymeric tubing which is placeable proximate to the respective arm 130a, 150a, FIGS. 7A-8.

When connected to a standard dental HVE suction source, device 100a, FIG. 8, preferably removes upwards of 90% of the aerosol, and hence viral pathogens, generated by dental operative or hygiene procedures. In some constructions, the bottom orifice of the device 100a is connected to a typical plastic dental HVE suction tip. The suction tip can be shortened if necessary, with scissors.

Proper use of the device 100a is critical for safe and effective dental treatment in the era of COVID-19. This device should only be used by, or under direct supervision of, a trained and licensed dental professional, referred to herein as a practitioner.

In one construction, at least the cheek retractors 120a, 140a are both sufficiently flexible and resilient to enable the practitioner to gently compress the retractor portions and insert them the patient's mouth, between upper jaw UJ and lower jaw LJ as illustrated in FIG. 8, after connection of the proximal end of device 100a to the HVE suction source. The lips of the patient are gently retracted by carefully releasing the cheek retractors 120a, 140a, which opens the operative field for access by the practitioner. Only then the dental HVE be started, slowly ramping up to full strength. In some constructions, the device comes with adjustable suction orifice plugs, which the dentist or hygienist can add or remove in order to obtain optimal aerosol suction.

Once finished with the dental procedure, please remove the device from the patient's mouth. Please discard the suction device, the plastic HVE tip, and any adjusting plugs. Multiple uses of the device is not recommended.

Yet another alternative construction according to the present invention, device 200, FIGS. 9A-14B, has at least one of the right-side passageway and/or the left-side passageway defined by the right-side arm 230 and/or the left-side arm 250, respectively; in other words, at least one of the arms may be hollow or otherwise contain the respective passageway. Flat section 231 may serve as a strut to add further support to arms 230, 250. Section 231 may also serve as a rest or stop against the lower lip or chin of a patient.

Device 200 has a central housing 202 having a proximal end 204 and a distal end 206. The proximal end 204 defines a proximal housing opening 205 and the distal end 206 defines a distal housing opening 207 having a first open area. The housing 202 defines at least one housing side port 210, 212 on each side of the housing within arms 230, 250 in this construction, such as ports 210 and 212 indicated in phantom in FIGS. 10B and 13B. The housing 202 also defines at least one internal passage 214 in fluid communication with the distal housing opening 207, the proximal housing opening 205, and the housing side ports 210, 212. The proximal end 204 of the housing is shaped to be connectable, such as by having an outer cylindrical sleeve surface 203, to a conduit for a source of negative pressure, particularly to a HVE source.

The device 200 further includes a right-side cheek retractor 220 and a left-side cheek retractor 240. Each retractor 220, 240 defines a concave buccal-side surface 222, 242, respectively, and a convex oral-side surface 224, 244, respectively. The oral-side surface 224, 244 of each cheek retractor includes an inner portion 264, 284 positionable toward the pharynx of the patient, a middle portion 263, 283, and an outer portion 262, 282, respectively, positionable toward a practitioner.

Each retractor 220, 240 defines a retractor chamber 226, 246, and at least a first arc 266, 286 of, retractor openings: (i) positioned along one of the portions 262, 263, 264 and 282, 283, 284 of the oral-side surface 224, 244, respectively; (ii) communicating with the retractor chamber 226, 246, respectively; and (iii) having a first distribution of total open area along that retractor 220, 240, respectively. In some constructions, the device further includes a second arc 270, 290 of retractor openings positionable closer to the pharynx of the patient and having a second distribution of total open area along that retractor that is smaller than the first distribution of total open area along that retractor. In one embodiment, the second arc 270, 290 of retractor openings extends along the inner portion 264, 284 of the oral-side surface 224, 244 of each cheek retractor 220, 240.

FIG. 15 is a schematic top view of a device according to the present invention defining a distal opening 307 and having a left-side retractor 310 defining an arc 312 of “ascending” openings 314, 316, 318, 320, 322 having progressively larger open areas and a right-side retractor 410 defining an arc 412 of “descending” openings 414, 416, 418, 420, 422 having progressively smaller open areas.

In some alternative constructions according to the present invention, at least one opening has an open area that is selectively adjustable, such as by providing at least one plug 440, FIG. 15B, that is selectively deployable to decrease the size of, or totally occlude, the open area of at least one opening 450. In another construction, a single-use punch-out 460, FIG. 15C having score lines SC extending partially through a retractor wall, is depressed to reveal or enlarge the opening. In yet other constructions, one or more slidable or rotatable orifice adjustments are provided for selective adjustment of effective open area.

### EXAMPLE 1

A polymeric central housing prototype 500, FIG. 16, was 3D printed (made by additive manufacturing) with proximal and distal openings 505, 507 and opposing side ports 510, 512. An adaptor (not shown) was made to connect tubing from the proximal opening 505 to a shop vacuum (ShopVac negative pressure equipment, six-gallon capacity, 3.0 HP motor, 143 CFM). Most dental HVE systems have a lower rating of around 100 CFM.

The side ports 510, 512 on the central housing prototype 500, FIG. 16, were connected to a pair of tubing 520, 540 to represent cheek retractors with openings according to the present invention for testing purposes. The tubing length on the prototype 500 matches the length of the arms and the tubing connection for device 100a, FIGS. 7A-8. Arms 530, 550, FIG. 16, were added to central housing 502 to support middle portions of tubing 520, 540, respectively. Plugs 513 514 were placed in the distal openings of the tubing 520, 540, respectively. Inner diameters ID and circular cross-sectional areas are listed in Table 1:

TABLE 1
Cross-Sectional Areas of Test Article

| Part | ID | Cross-Sectional Area |
|---|---|---|
| Main tube | 5/16″ | 0.077 |
| Tubing Adapter | 0.23″ | 0.083 |
| Connector tubing and Arms | ¼″ | 0.098 |

FIG. 17 depicts known preferred relationships among cross-sectional areas of branching passageways, beginning with primary central passageway A which branches into secondary passageways B₁ and B₂. In turn, secondary passageway B₁ branches into tertiary passageways C₁ and C₂, while secondary passageway B₂ branches into tertiary passageways. C₃ and C₄. Preferably, the sum of the cross-sectional areas of each branch should be equal to or greater than the cross-sectional areas of the proceeding branches; the minimum “equal to” relationship is expressed by Equation 1:

$$A = ( {B}_{1} + {B}_{2} ) = ( {C}_{1} + {C}_{2} + {C}_{3} + {C}_{4} ) \qquad Eq . 1$$

Inner diameters ID for the 3D printed central housing prototype 500, FIG. 16, including ports 510, 512 and tubing 520, 540, were chosen to minimize restrictions in branching passageways therein as shown above in Table 1.

For Example 1, the central housing and a continuous aerosolizer 501 were placed on test stands and spaced from each other at a distance D as schematically illustrated in FIGS. 18A-18C. A reservoir of water with blue-color food-grade dye was connected to the aerosolizer 501. The central distal hole 507 of the central housing 502 was 9 mm in diameter. This 9 mm ID is equivalent to the ID of the distal opening of a standard HVE tip. Therefore, the central housing 502 was utilized without added tubing as a baseline sample to represent conventional HVE devices. The tubing adapter ports 510, 512 were plugged for “conventional” use as shown in FIG. 18A with aerosolizer 501 generating aerosol clouds 508_{A}, 508_{E} and 508_{C} in FIGS. 18A-C, respectively. Tests were conducted at two distances D of 5.5 inches and 12 inches.

Three patterns were tested where the combined cross-sectional area was lower than that of the proceeding branches confirm that this pattern is less effective, as listed in the last three rows in Table 2 below. Preferably, the combined cross-sectional area of the holes should be greater than the combined cross-sectional area of the proceeding branch (≥0.098 in²).

It was determined that, for an arc of constant-diameter holes, suction power decreases distally along the length of the arms. For an arc of ascending diameter holes, increasing the diameter along the length of the arms increases CFM but decreases suction power towards the end of the arms; as shown in FIG. 18B, a portion of aerosol cloud 508_{B} escaped past the tubing 520, 540. By comparison, for an arc of descending diameter holes, decreasing the diameter along the length of the arms decreases CFM but increases suction power along the length of the arms, which improves aerosol capture as represented by captured aerosol cloud 508c, FIG. 18C.

In summary, a descending hole pattern, proceeding distally along the cheek retractors, was found to be more effective at capturing aerosol than having a constant hole size or an ascending hole pattern. When an aerosol cloud is focused and non-dispersed, the descending hole pattern performed comparably to a conventional HVE device. However, when the aerosol cloud is larger and more dispersed, which is more likely to occur with actual patients, then the descending hole pattern performed better than the HVE-equivalent as tested.

TABLE 2
Hole Patterns tested and their cross-sectional areas

| | | Cross | |
|---|---|---|---|
| | | Sectional | |
| Pattern | | Area | Hole |
| Type | Description | (in\hat{ }2) | Quantity |
| Ascending | Holes were 2-5 mm, increasing 0.5 | 0.113 | 7 |
| | mm along the length of the arm so | | |
| | the largest hole is at the top. The | | |
| | holes were spaced evenly. | | |
| 3.5 mm | 3.5 mm holes were spaced evenly | 0.104 | 7 |
| holes | along the arms. | | |
| Descending | Holes were 2-5 mm, decreasing 0.5 | 0.113 | 7 |
| | mm along the length of the arm so | | |
| | the smallest hole is at the top. The | | |
| | holes were spaced evenly. | | |
| Descending | Holes were 3-5 mm, decreasing 0.5 | 0.100 | 5 |
| | mm along the length of the arm so | | |
| | the smallest hole is at the top. The | | |
| | holes were spaced evenly. | | |
| 5 mm | 5 mm holes were spaced evenly | 0.091 | 3 |
| holes | along the arm. | | |
| 5 mm | 5 mm holes were spaced evenly | 0.061 | 2 |
| holes | along the arm. The bottom hole | | |
| | was plugged. | | |
| 5 mm | 5 mm holes were spaced evenly | 0.030 | 1 |
| holes | along the arm. The two bottom | | |
| | holes were plugged. | | |

It is currently preferred to have openings in a descending hole pattern, with holes positioned toward the middle portion of each cheek retractor.

A list of reference numerals utilized to describe device 100, FIGS. 1A-6B, is provided below. Equivalent numerals utilized in other Figures represent equivalent features unless indicated otherwise.

### LIST OF REFERENCE NUMERALS

### 100 Device, FIGS. 1A-6B

102 central housing
103 outer cylindrical sleeve surface
104 proximal end
105 proximal housing opening
106 distal end
107 distal housing opening having a first open area
110 right-side housing side port
112 left-side housing side port
114 internal passage of housing 102
120 right-side cheek retractor
122 concave buccal-side surface
124 convex oral-side surface
162 outer portion of 124 positionable toward a practitioner
163 middle portion of 124
164 inner portion of 124 positionable toward the pharynx of the patient
166 first arc of openings 168
168 openings of first arc 166
170 second arc of openings 172
172 openings of second arc 170
126 retractor chamber
129 connector
130 right-side arm
140 left-side cheek retractor
142 concave buccal-side surface
144 convex oral-side surface
182 outer portion of 144 positionable toward a practitioner
184 inner portion of 144 positionable toward the pharynx of the patient
186 first arc of openings 188
188 openings of first arc 186
190 second arc of openings 192
192 openings of second arc 190
146 retractor chamber
149 connector
150 left-side arm

### For FIGS. 7A-8 (Device 100a et seq.):

128a right-side passageway
148a left-side passageway
165a, 185a Middle sections of middle portions 163a, 183a

In yet another construction, a single member, or stem, which inserts onto a traditional dental HVE line. It forms a “T” at the base of the cheek-retractor portion of the device. It has an HVE opening at the base of this “T” portion. The above described are made of a semi-rigid, hollow plastic. The device then transitions into the cheek-retractor portion. This is also made from a hollow, semi-rigid plastic. However, it will be of a more flexible type of plastic then the base, or “T” portion.” The cheek-retractor portion is composed of two semi-lunar sections of plastic which are cupped to contain the lips/cheeks within. It will have HVE holes located around the periphery of the oral cavity, ranging from small holes near the base to large holes at the terminal ends. It is the size, distribution, and arrangement of holes which provide the “broad field” suction.

This invention may also be expressed as a device suitable for evacuating aerosols generated during dental procedures, including a housing having a proximal end and a distal end with a distal housing opening having a first cross-sectional area, and the housing defining at least one internal passage in fluid communication with the distal housing opening and at least one housing port, the at least one housing port adapted to be connected to a source of negative pressure. A right-side cheek retractor and a left-side cheek retractor extend from the housing, each retractor defining an outer arc of holes having a second cross-sectional area. In certain constructions, at least one retractor includes an inner arc of holes having a third average cross-sectional area, the third average diameter being smaller than the second average diameter. The term “average cross-sectional area” as utilized herein refers to the sum of cross-sectional areas of each opening along an arc of holes divided by the number of such openings along that arc, and includes irregularly-shaped and non-circular openings.

Although specific features of the present invention are shown in some drawings and not in others, this is for convenience only, as each feature may be combined with any or all of the other features in accordance with the invention. While there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also fully intended and contemplated. It is also to be understood that the drawings are not necessarily drawn to scale, but that they are merely conceptual in nature.

It is the intention, therefore, to be limited only as indicated by the scope of the claims appended hereto. Other embodiments will occur to those skilled in the art after reviewing the present disclosure and are within the following claims.

What is claimed is:

1. A device suitable for evacuating aerosols generated proximate to teeth of a patient during dental procedures, comprising:
a central housing having a proximal end and a distal end, the proximal end defining a proximal housing opening and the distal end defining a distal housing opening having a first open area, and the housing defining (i) at least one housing side port on each side of the housing and (ii) at least one internal passage in fluid communication with the distal housing opening, the proximal housing opening, and the housing side ports, the proximal end of the housing shaped to be connectable to a conduit for a source of negative pressure;
a right-side cheek retractor and a left-side cheek retractor, each retractor defining a concave buccal-side surface, a convex oral-side surface, a retractor chamber, and at least a first arc of retractor openings (i) positioned along a portion of the oral-side surface, (ii) communicating with the retractor chamber, and (iii) having a first distribution of total open area along that retractor;
a right-side arm extending between the housing and the right-side cheek retractor to support the right-side cheek retractor at a substantially fixed right-side distance from the housing;
a left-side arm extending between the housing and the left-side cheek retractor to support the left-side cheek retractor at a substantially left-side fixed distance from the housing;
at least one right-side passageway connecting one of the housing side ports with the retractor chamber of the right-side cheek retractor; and
at least one left-side passageway connecting one of the housing side ports with the retractor chamber of the left-side cheek retractor.

2. The device of claim 1 wherein at least one of the right-side passageway and/or the left-side passageway is defined by tubing.

3. The device of claim 1 wherein at least one of the right-side passageway and/or the left-side passageway is defined by the right-side arm and/or the left-side arm, respectively.

4. The device of claim 1 wherein (i) the oral-side surface of each cheek retractor includes (a) an inner portion positionable toward the pharynx of the patient, (b) an outer portion positionable toward a practitioner, and (c) a middle portion disposed between the inner portion and the outer portion, and (ii) the first arc of retractor openings extends along at least one of the outer portion and the middle portion of the oral-side surface of each cheek retractor.

5. The device of claim 4 wherein at least one of the arcs of retractor openings has openings that decrease in size progressing away from the distal housing opening.

6. The device of claim 4 further including a second arc of retractor openings positionable closer to the pharynx of the patient and having a second distribution of total open area along that retractor that is smaller than the first distribution of total open area along that retractor.

7. The device of claim 6 wherein the second arc of retractor openings extends along the inner portion of the oral-side surface of each cheek retractor.

8. The device of claim 1 wherein each retractor and each arm is formed of a substantially resilient material.

9. A method for evacuating aerosols generated proximate to teeth in a mouth of a patient during dental procedures, comprising:
selecting a device including a central housing having a proximal end and a distal end, the proximal end defining a proximal housing opening and the distal end defining a distal housing opening having a first open area, and the housing defining (i) at least one housing side port on each side of the housing and (ii) at least one internal passage in fluid communication with the distal housing opening, the proximal housing opening, and the housing side ports, the proximal end of the housing shaped to be connectable to a conduit for a source of negative pressure;
wherein the device further includes a right-side cheek retractor and a left-side cheek retractor, each retractor defining a concave buccal-side surface, a convex oral-side surface, a retractor chamber, and at least a first arc of retractor openings (i) positioned along a portion of the oral-side surface, (ii) communicating with the retractor chamber, and (iii) having a first distribution of total open area along that retractor, with a right-side arm extending between the housing and the right-side cheek retractor to support the right-side cheek retractor at a substantially fixed right-side distance from the housing, and a left-side arm extending between the housing and the left-side cheek retractor to support the left-side cheek retractor at a substantially left-side fixed distance from the housing;
wherein at least one right-side passageway connects one of the housing side ports with the retractor chamber of the right-side cheek retractor, and at least one left-side passageway connects one of the housing side ports with the retractor chamber of the left-side cheek retractor;
positioning the device such that the buccal-side surface of the right-side cheek retractor contacts a left cheek of the patient and the buccal-side surface of the left-side cheek retractor contacts a right cheek of the patient, with the distal housing opening positioned below the mouth of the patient; and
applying negative pressure to the proximal housing opening to evacuate aerosols, generated proximate to the teeth of the patient during a dental procedure, through the retractor openings and the distal housing opening.

10. The method of claim 9 wherein (i) the oral-side surface of each cheek retractor includes (a) an inner portion positionable toward the pharynx of the patient, (b) an outer portion positionable toward a practitioner, and (c) a middle portion disposed between the inner portion and the outer portion, and (ii) the first arc of retractor openings extends along at least one of the outer portion and the middle portion of the oral-side surface of each cheek retractor.

11. The method of claim 10 wherein at least one of the arcs of retractor openings has openings that decrease in size progressing away from the distal housing opening.
